(19) United States
(12) Patent Application Publication
(10) Pub. No.: US 2007/0106368 A1
(43) Pub. Date: May 10, 2007

(54) Graft-stent assembly

(76) Inventor: Carlos Vonderwalde, Richmond (CA)

Correspondence Address: Martin Moynihan;PRTSI, Inc., P.O. Box 16446, Arlington, VA, 22215, US

(21) Appl. No.: 11/267,313

(22) Filed: Nov. 7, 2005

(51) Int. Cl. A61F 2/06

(52) U.S. Cl. 623/1.13

(57) ABSTRACT

A graft-stent assembly comprising a stent and a substantially tubular graft branching therefrom is disclosed. The stent is preferably deployed in a trunk vessel to support trunk vessel patency while the tubular graft is preferably deployed in a branching vessel. In embodiments, the downstream end of the tubular graft is provided with an expandable ring-shaped support member to anchor the downstream end of the tubular graft in place.

### FIELD AND BACKGROUND OF THE INVENTION

The present invention relates to the field of surgery and more particularly, to a method and a device useful for maintaining patency of a bifurcated lumen especially of the cardiovascular system.

A stent is a device deployed inside a lumen of a bodily vessel to physically maintain patency of the vessel. Typical vessels treated with stents include respiratory ducts, gastrointestinal ducts, lymphatic ducts, blood vessels and especially arteries that are occluded, stenosed, aneuritic, physically damaged, diseased, collapsing or weakened.

Stents are usually outwardly radially expandable, having a substantially tubular shape both in an unexpanded state with a small radial dimension and in any one of the expanded states with larger radial dimensions. Various constructions of stents are known including rolled-up sheets, slotted or otherwise cut-out tubes and bent wires.

For deployment inside a lumen of a bodily vessel an expandable stent is placed in an unexpanded state on a deployment catheter, inserted through an incision in the skin and maneuvered through the body to the deployment location. The stent is then expanded to an appropriately-sized expanded state so as to engage the inner walls of the treated vessel and to thus maintain patency thereof.

A first type of stent is the self-expanding stent. When the stent is at the deployment location, the stent is released from the catheter and allowed to expand to an expanded state, in a manner analogous to that of a compressed spring. Self-expanding stents have been disclosed, for example, in U.S. Pat. Nos. 4,503,569; 4,580,568; 4,787,899; and 5,104,399.

A second type of stent is expanded from the unexpanded state to an expanded state using an expansion device, typically a catheter-borne balloon. When the stent is at the deployment location, the expansion device is activated inside the bore of the unexpanded stent to exert an outwards radial force on the inside of the stent, causing the stent to expand to a desired size. Such stents have been disclosed, for example, in U.S. Pat. Nos. 4,655,771; 4,733,665; 4,739,762; 4,800,882; 4,907,336; 4,994,071; 5,019,090; 5,035,706; 5,037,392; and 5,147,385.

As is known to one skilled in the art, many bodily vessels are bifurcated. By “bifurcated” is meant an object that splits to two branches along a length of the object. Two types of bifurcated objects (bifurcated blood vessels) having two different types of bifurcation are depicted in FIGS. 1A and 1B. A first type of bifurcated object 10 depicted in FIG. 1A includes a trunk vessel 12 from which a branch vessel 14 branches downstream from a bifurcation point 16. Generally, but not necessarily, the bore of branch vessel 14 is smaller than that of trunk vessel 12. A second type of bifurcated object 18 depicted in FIG. 1B includes a trunk vessel 12 from which two branch vessels 14A and 14B branch downstream from bifurcation point 16. Generally, but not necessarily, the bores of branch vessels 14A and 14B are smaller than the bore of trunk vessel 12. Branch vessel 14B of bifurcated object 18 is also bifurcated: branch vessel 14B is a trunk vessel from which branch vessel 20 branches. Although many naturally occurring vessels can be clearly classified as having either the first or the second type of bifurcation there are cases where such classification is ambiguous as there is no exact delineation of characteristics distinguishing one of the two types of bifurcation from the other.

In the field of medicine it is known to deploy stents in a bifurcated bodily vessel. Often, both branches of a bifurcated bodily vessel must be treated as damage, lesions or athersclerotic disease are often found in both the trunk and the branch vessels.

In some instances, it is prefered to deploy a stent in a healthy branch vessel together with deployment of a stent in a pathological trunk vessel, see for example U.S. Pat. Nos. 4,994,071; 5,669,924; 5,723,004; 5,906,640 or PCT patent application No. PCT/IB98/00496 published as WO 99/15103 of the inventor.

In some instances where the trunk vessel of a bifurcated vessel is damaged but the branch vessel is substantially healthy it is preferable not to deploy a stent in the branch vessel to avoid the physical stress caused to the branch vessel by the deployment of the stent as well as to avoid the reduction of the bore size of the branch vessel necessarily caused by stent deployment. It is therefore known to deploy a stent only in the trunk vessel. However, often parts of the stent deployed in the trunk vessel partially obstruct the entrance into the branch vessel, reducing flow rate into the branch vessel, increasing pressure at the bifurcation point and causing turbulent flow, factors that may lead to restenosis of the trunk vessel and stenosis of the branch vessel. It is known to deploy a stent having a side port in the trunk vessel so as to reduce obstruction of the branch vessel. However, interventional manipulation of the trunk vessel often compromises the integrity of the branch vessel or bifurcation point whether the stent deployed in the trunk vessel has a side port or not.

In some instances, both the trunk vessel and the branch vessel are damaged, diseased and in need of support but the structural integrity of the branch vessel is compromised to the point that there is the fear that stent deployment will lead to catastrophic failure of the branch vessel. In such cases there is little choice but to invasively replace the branch vessel.

It would be advantageous to have a stent assembly that supports a trunk vessel and provides a measure of structural support for a branch vessel without requiring deployment of a stent in the branch vessel. Ideally, such a stent assembly would:
a) function as an effective stent for supporting the trunk vessel;
b) cause minimal or no damage to both the bifurcation point or branch vessel during deployment;
c) provide sufficient support to the bifurcation point and branch vessel if damaged during deployment to prevent fluid leakage and vessel bursting;
d) provide a smooth and unobstructed flow path from the trunk lumen into the branch vessel to allow unimpeded and non-turbulent flow;
e) be flexible so that a given stent assembly is deployable in a wide range of bifurcated vessels having branch vessels of different sizes and diverging at many different angles from a trunk vessel; and
g) lack rigidity so that once deployed the orientation of the branch implant and trunk implant of the stent assembly would change together with the natural movement of the organ (e.g., beating of the heart) in which deployed to prevent structural stress of the bifurcated vessel.

It would be highly advantageous to have a stent assembly useful for deployment in bifurcated vessels and not having at least some of the disadvantages of the prior art.

### SUMMARY OF THE INVENTION

The present invention successfully addresses at least some of the shortcomings of prior art by providing a graft-stent assembly for deployment in a bifurcated bodily vessel.

The present invention is of a graft-stent assembly including a trunk stent or trunk stents for deployment in a trunk vessel and a substantially tubular graft for deployment in a branch vessel.

When deployed, the trunk stent or stents of a graft-stent assembly of the present invention maintain patency of the trunk vessel. Preferably, the graft is deployed in the branch vessel, providing a smooth transition into the branch vessel and maintaining patency of the branch vessel with little physical stress. Embodiments of the present invention allow quick and simple relining of a branch vessel. The branch graft of embodiments of the present invention is easily trimmed to shorten the length of the branch graft for deployment. In embodiments of the present invention, the branch graft easily bends to conform to a branch vessel in which implanted.

According to the teachings of the present invention there is provided a graft-stent assembly, comprising: a) a flexible graft including a substantially tubular graft wall, a proximal graft end, a distal graft end and a graft bore; and b) a substantially tubular first expandable stent having a first end, a second end and a bore in fluid communication with the graft bore through the proximal graft end; wherein the graft bore and the bore of the first stent are discontinuous. Preferably, the graft bore substantially defines a branch part of the graft-stent assembly and the bore of the first stent defines, at least in part, a trunk part of the graft-stent assembly.

In an embodiment of the present invention, the graft bore diverges from the bore of the first stent at an angle of greater than about 5°, greater than about 10° or even greater than about 30°.

In an embodiment of the present invention, the first stent is secured to the graft, for example, by sutures, adhesives, bending members, clamps, glue, hooks, piercing members, staples, laser welding other applicable mechanical mean or combinations thereof.

In an embodiment of the present invention, the graft-stent assembly further comprises: c) an expandable ring-shaped support member functionally associated with the graft through the distal graft end, and is configured to radially expand from an unexpanded state. Preferably the graft bore and the ring-shaped support member substantially define a branch implant of the graft-stent assembly and the bore of the first stent substantially defines a trunk implant of the graft-stent assembly.

In an embodiment of the present invention, a part of the graft wall is threaded through the ring-shaped support member. In an embodiment of the present invention, the distal graft end is threaded through the ring-shaped support member and folded thereover.

In an embodiment of the present invention, the ring-shaped support member is substantially entirely contained within the graft bore. In an embodiment of the present invention, the ring-shaped support member is located inside the bore of the graft wall and the distal graft end is folded thereover. In an embodiment of the present invention the distal graft end does not extend beyond the ring-shaped support member. In an embodiment of the present invention, the ring-shaped support member does not significantly extend beyond the distal graft end.

In an embodiment of the present invention, the axial length of the ring-shaped support member is substantially shorter than the axial length of the graft wall. In embodiments of the present invention, the axial length of the ring-shaped support member is less than about 30% and even less than about 10% of the axial length of the graft wall.

In an embodiment of the present invention, the ring-shaped support member is secured to the distal graft end, preferably at a plurality of locations about the circumference of the distal graft end, for example, using sutures, adhesives, bending members, clamps, glue, hooks, piercing members, staples, laser welding other applicable mechanical mean or combinations thereof. In an embodiment of the present invention, the ring-shaped support member is configured to clamp over the distal graft end at a plurality of locations on the distal graft end. In an embodiment of the present invention, a part of the ring-shaped support member is bent about the distal graft end so as to clamp the distal graft end.

In an embodiment of the present invention, the graft-bore diverges from a side of the first stent. In such an embodiment, preferably the graft-bore substantially defines a branch implant of the graft-stent assembly and the bore of the first stent substantially defines a trunk implant of the graft-stent assembly. Preferably, the first stent is provided with a side opening through which the graft bore is in fluid communication with the bore of the first stent.

In an embodiment of the present invention, the graft is connected to the first stent with at least one, preferably at least two, connecting tab or tabs, the connecting tab or tabs contacting the outer surface or the inner surface of the stent. In embodiments of the present invention, a connecting tab is integrally formed with the flexible graft. In embodiments of the present invention, a connecting tab is secured to the flexible graft, for example, using sutures, adhesives, bending members, clamps, glue, hooks, piercing members, staples, laser welding other applicable mechanical mean or combinations thereof. In an embodiment of the present invention, a connecting tab or tabs is secured to the first stent, for example, with the help of sutures, adhesives, bending members, clamps, glue, hooks, piercing members, staples, laser welding other applicable mechanical mean or combinations thereof.

In an embodiment of the present invention, a graft of a graft-stent assembly of the present invention further comprises a second substantially tubular graft part having a bore not parallel to the graft bore connecting the graft to the first stent.

In an embodiment of the present invention, the second substantially tubular graft part surrounds at least part of the first stent. In an embodiment of the present invention, the first stent is substantially entirely contained within the second substantially tubular graft part. In an embodiment of the present invention, at least one end of the first stent emerges from the second substantially tubular graft part. In an embodiment of the present invention, the two ends of the first stent emerge from the second substantially tubular graft part.

In an embodiment of the present invention, at least part of the second substantially tubular graft part is disposed within the bore of the first stent. In an embodiment of the present invention, the second substantially tubular graft part is substantially entirely disposed within the bore of the first stent. In an embodiment of the present invention, a first end of the second substantially tubular graft part emerges from the first end of the first stent. In an embodiment of the present invention, a first end of the second substantially tubular graft part emerges from the first end of the first stent and is folded thereover. In an embodiment of the present invention, a second end of the second substantially tubular graft part emerges from the second end of the first stent. In an embodiment of the present invention, a second end of the second substantially tubular graft part emerges from the second end of the first stent and is folded thereover.

In an embodiment of the graft-stent assembly of the present invention, the graft includes a second substantially tubular graft part having a first end, a second end and a bore not parallel to the graft bore connecting the graft to the first stent through the first end of the second substantially tubular graft part, and further comprising: d) a second expandable stent having a first end, a second end and a bore in fluid communication with the graft bore connected to the graft through the second end of the second substantially tubular graft part. In such an embodiment, preferably the graft bore substantially defines a branch implant of the graft-stent assembly and the bore of the first stent, the bore of the second stent and the bore of the second substantially tubular graft part substantially define a trunk implant of the graft-stent assembly.

In an embodiment of the present invention, the second substantially tubular graft part surrounds at least part of the first stent. In an embodiment of the present invention, the first stent is substantially entirely contained within the second substantially tubular graft part. In an embodiment of the present invention, the second end of the first stent emerges from the second substantially tubular graft part. In an embodiment of the present invention, the second substantially tubular graft part surrounds at least part of the second stent. In an embodiment of the present invention, the second stent is substantially entirely contained within the second substantially tubular graft part. In an embodiment of the present invention, the second end of the second stent emerges from the second substantially tubular graft part.

In an embodiment of the present invention, at least part of the second substantially tubular graft part is disposed within the bore of the first stent. In an embodiment of the present invention, a first end of the second substantially tubular graft part emerges from the second end of the first stent. In an embodiment of the present invention, a first end of the second substantially tubular graft part emerges from the second end of the first stent and is folded thereover. In an embodiment of the present invention, at least part of the second substantially tubular graft part is disposed within the bore of the second stent. In an embodiment of the present invention, a first end of the second substantially tubular graft part emerges from the second end of the second stent. In an embodiment of the present invention, a first end of the second substantially tubular graft part emerges from the second end of the second stent and is folded thereover.

In an embodiment of the of the present invention, the graft-stent assembly further comprises: c) an expandable ring-shaped support member (as described above) functionally associated with the graft through the distal graft end, the ring-shaped support member configured to radially expand from an unexpanded state. In such an embodiment, preferably the graft bore and the ring-shaped support member substantially define a branch implant of the graft-stent assembly and the bore of the first stent, the bore of the second stent and the bore of the second substantially tubular graft part substantially define a trunk implant of the graft-stent assembly.

In an embodiment of the present invention, the graft wall is substantially elastic.

In an embodiment of the present invention, the graft wall is substantially impervious to tissue proliferation therethrough. Such imperviousness is useful in preventing tissue buildup on and through the graft wall and prevents the migration of smooth muscle cells. Such an embodiment is useful for providing a treated bodily vessel with a new, undamaged, smooth lining that is substantially impervious to restenosis.

In an embodiment of the present invention, the graft wall is substantially impermeable to fluids.

In an embodiment of the present invention, the graft wall is permeable. Such an embodiment is useful as the permeability allows cells to grow into and through the graft wall, making the graft wall substantially part of the vessel in which deployed.

Generally, the walls of a graft of the present invention are as thin as possible to ensure that a respective graft-stent assembly be flexible, have a low profile during navigation to the deployment site and to restrict a vessel in which deployed as little as possible and at the same time the walls must be sufficiently elastic and strong to permit navigation and deployment without tearing. Clearly, the nature of the material from which a given graft is made determines in part the thickness of the walls. That said, a graft preferably has walls that are not thicker than about 0.75 mm, not thicker than about 0.45 mm, not thicker than about 0.25 mm, not thicker than about 0.20 mm, and even not thicker than about 0.05 mm.

In an embodiment of the present invention, the graft and especially the graft wall are substantially fashioned from a synthetic or polymeric material including but not limited to materials such as polytetrafluoroethylene, urethane, elastomer, polyamide (e.g., Nylon) and polyester (e.g., Dacron).

In an embodiment of the present invention, the graft and especially the graft wall are substantially fashioned from a material which is biological tissue including but not limited to autologous tissue, heterologous tissue, venous tissue, arterial tissue, serous tissue, pleura, peritoneum, pericardium and aortic leaflet. In an embodiment of the present invention, the material is harvested from a source selected from the group consisting of human sources and non-human animal sources, especially equine, porcine, bovine and human. In an embodiment of the present invention the material is thinned, where after harvesting one or more layers of the harvested tissue is removed, e.g., by scraping, shaving, slicing or skiving. In an embodiment of the present invention, the material (or components of the material such as collagen) is cross-linked, for example by treatment with a glutaraldehyde or a phosphate solution.

In an embodiment of the present invention, the graft and especially the graft wall are substantially fashioned from serous membrane including a serous tissue stratum and a basement tissue stratum, where the serous membrane is, for example, from porcine, bovine, equine or human serous membrane.

In a preferred embodiment of the present invention, the graft and especially the graft wall are substantially fashioned from a thinned serous membrane, where a harvested serous membrane (peritoneum, pericardium or pleural tissue especially porcine, bovine, equine and human serous tissue) has been processed by removal of a layer of at least some of the associated basement tissue (and thus thinned), preferably removal of all the basement tissue, leaving only the serous tissue layer. Thus in an embodiment of the present invention, the graft and especially the tubular graft wall comprises a thinned serous membrane including a serous tissue stratum and a basement tissue stratum, wherein the thinned serous membrane has been processed by removal of a layer of basement tissue from a harvested serous membrane. In an embodiment of the present invention only a layer of the basement tissue is removed. In an embodiment of the present invention, the thinned serous membrane is substantially serous tissue devoid of basement tissue. In an embodiment of the present invention, the material consists essentially of serous tissue.

When serous tissue is used to implement the teachings of the present invention it is usually preferred to orient the membrane so that the smooth serous strata is facing the fluid flow to reduce turbulent flow.

In embodiments of the present invention, one or more of the component stents of the graft-stent assembly of the present invention are jacketed, for example with any of the stent jackets known in the art.

In embodiments of the present invention, one or more of the component stents of the graft-stent assembly of the present invention are coated, for example with any of the stent coatings known in the art.

Expandable ring-shaped support members suitable for use in implementing the teachings of the present invention are taught in PCT Patent Application No. IB01/00315 published as WO01/66037 of the inventor. Expandable ring-shaped support members suitable for implementing the teachings of the present invention may be self-expanding or non self-expanding. Generally, self-expanding ring-shaped support members are preferred. Two important parameters used when selecting a stent for use in implementing the teachings of the present invention are the expanded and unexpanded diameters of the stent.

Generally it is important that the unexpanded diameter of a stent be as small as possible to ease navigation through the body to the deployment location. That said, the unexpanded diameter must be large enough to allow threading of the stent onto a deployment catheter and, for not-self expanding stents, onto a stent-expanding device such as a stent-expanding balloon. Although there may be some variation in the unexpanded diameter of even two identical stents depending on how the two stents are used, herein by unexpanded diameter is intended the outer diameter of an expandable stent when crimped to the smallest practical diameter onto a delivery catheter for deployment.

As noted above embodiments of a graft stent of the present invention include two stents associated end to end by a second tubular graft part with the tubular graft wall itself substantially branching off from the space between the ends of the two stents. Generally, the distance between the ends of the stents is relatively small, but not smaller than the diameter of the proximal end of the tubular graft wall so as not to obstruct fluid flow from the bore of the stents into the tubular graft wall. That said, in embodiments of a graft-stent of the present invention the distance between the ends of the first expandable stent and the second expandable stent is generally not greater than the diameter of the proximal end of the tubular graft wall, not greater than twice the diameter of the proximal end of the tubular graft wall and not greater than four times the diameter of the proximal end of the tubular graft wall.

Further, in embodiments of a graft-stent of the present invention comprising two stents, the distance between the ends of the first expandable stent and the second expandable stent is generally no greater than about four, no greater than about three, no greater than about two, no greater than about one and even no greater than about half of an unexpanded diameter of the second expandable stent.

In embodiments of the present invention, a first expandable stent and a second expandable stent (if present) are of substantially similar or identical dimensions, especially length, expanded diameter and/or unexpanded diameter. In embodiments of the present invention a first expandable stent and a second expandable stent (if present) are of substantially different dimensions, especially length, expanded diameter and/or unexpanded diameter. In embodiments of the present invention, the diameter (expanded or unexpanded) of the first expandable stent is substantially similar to the respective diameter of the second expandable stent (if present). In embodiments of the present invention, the diameter (expanded or unexpanded) of the first expandable stent is larger than the respective diameter of the second expandable stent (if present).

Generally, any given stent has a wide range of expanded diameters larger than a respective unexpanded diameter. The expanded diameter of a stent subsequent to deployment is determined by the user of the stent according to medical criteria including the natural size of the lumen of the vessel in which the stent is deployed. That said, self-expanding stents are characterized by a specific maximal expansion that is the maximal diameter of the stent when the stent is free from externally applied forces. Most non-self expanding stents are also characterized by a maximal expansion that is the greatest extent to which the stent is expandable without comprising the structural integrity thereof.

According to the teachings of the present invention there is also provided a method of preparing a graft-stent of the present invention, generally comprising providing an appropriate graft, a required number of stents and expandable ring-shaped support members and assembling the components in accordance with the description and the figures herein. In embodiments of the present invention, tubular components of a graft are made of substantially tubular tissue harvested as is.

In embodiments of the present invention, a graft comprises conjoined tubular components. In embodiments of the present invention, one or more tubular components are seamless, for example of harvested tubular tissue. In embodiments of the present invention, bifurcated tubular components of a graft are made of harvested substantially bifurcated tubular tissue harvested. In embodiments of the present invention, tubular components of a graft are made of substantially planar sheet of material that is fashioned into a tube, for example by overlapping or abutting two edges of the sheet. In embodiments of the present invention, a tubular graft wall of a graft is made of a first material while other components (e.g., connecting tabs or a second tubular graft part) are made of a second material.

In an embodiment of the present invention, a bifurcated graft of the present invention is essentially fashioned from one sheet, preferably planar or substantially planar, of an appropriate membrane (synthetic or harvested) rolled up into shape (with abutting or overlapping edges) and then fixed in shape using any of the methods known in the art. Preferred shapes of sheets suitable for use in fashioning a bifurcated graft from one sheet of material generally include shapes having at least one C2 symmetry axis and/or at least one external angle of no greater than 90° and/or at least 7 sides. Suitable shapes include cross shapes, “T”-shapes, “W”-shapes, “X”-shapes, “Y”-shapes and “Ψ”-shapes.

According to the teachings of the present invention there is also provided a method of treatment, for example of an aneurism, generally comprising deploying a graft-stent of the present invention inside the lumen of a bifurcated bodily vessel, especially in the cardiovascular, cerebrovascular or peripheral vascular system. Such methods of treatment include relining a vessel, supporting patency of a vessel, treating an aneurism or avoiding bursting of a vessel.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B are depictions of bifurcated objects in which deployment of a graft-stent assembly of the present invention is useful;

FIG. 2 is a depiction of a bifurcated artery with an aneurism on the branch vessel in which a graft-stent assembly of the present invention is deployed;

FIGS. 3A-3D depict distal ends of tubular graft walls of graft-stents of the present invention provided with expandable ring-shaped support members;

FIGS. 4A-4F depict graft-stents of the present invention including only one stent;

FIGS. 5A-5F depict graft-stents of the present invention including two stents;

FIGS. 6A and 6B depict stent-grafts of the present invention deployed in bifurcated arteries; and

FIGS. 7A-7G depict sheets of various shapes rolled-up and edges attached to fashion bifurcated grafts of the present invention.

### DESCRIPTION OF EMBODIMENTS OF THE INVENTION

The present invention is of a graft-stent assembly useful for deployment in bifurcated bodily vessels.

The principles, uses and implementations of the teachings of the present invention may be better understood with reference to the accompanying description and figures. Upon perusal of the description and figures present herein, one skilled in the art is able to implement the teachings of the present invention without undue effort or experimentation. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of medicine, biology, chemistry, material sciences and engineering. Such techniques are thoroughly explained in the literature. Unless otherwise defined, all technical and scientific terms used herein have the same meaning as commonly understood by one of ordinary skill in the art to which the invention belongs. In addition, the descriptions, materials, methods and examples are illustrative only and not intended to be limiting. Methods and materials similar or equivalent to those described herein can be used in the practice or testing of the present invention. All publications, patent applications, patents and other references mentioned are incorporated by reference in their entirety as if fully set forth herein. In case of conflict, the specification herein will control.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the relevant arts. Implementation of the methods of the present invention involves performing or completing selected tasks or steps manually, automatically, or a combination thereof.

The present invention is related to a bifurcated stent assembly useful for deployment in bifurcated bodily vessels including a trunk vessel and a branch vessel. The teachings of the present invention are based on the use of a graft-stent assembly including a trunk stent or trunk stents for deployment in a trunk vessel and a graft including a substantially tubular graft wall for deployment in a branch vessel. When deployed, the trunk stent or stents of a graft-stent assembly of the present invention maintain patency of the trunk vessel as known in the art while the graft is deployed in the branch vessel, providing a smooth transition into the branch vessel and maintaining patency of the branch vessel without physical stress. Further, the graft physically supports the vicinity of the bifurcation point of the bifurcated bodily vessel. Such support prevents the collapse of the bifurcated vessel in the vicinity of the bifurcation point. Further, if during stent deployment and expansion the walls of the bifurcated vessel in the vicinity of the bifurcation become weakened, torn or otherwise damaged the graft of the graft-stent assembly acts to contain fluids flowing through the bodily vessel and prevents the fluids breaking through the thus-damaged bodily vessel. Embodiments of the present invention allow quick and simple relining of a branch vessel. The tubular graft wall of embodiments of grafts of the present invention are easily trimmed to shorten the length of the branch graft for deployment. In embodiments of the present invention, the tubular graft wall easily bends to conform to a branch vessel in which deployed.

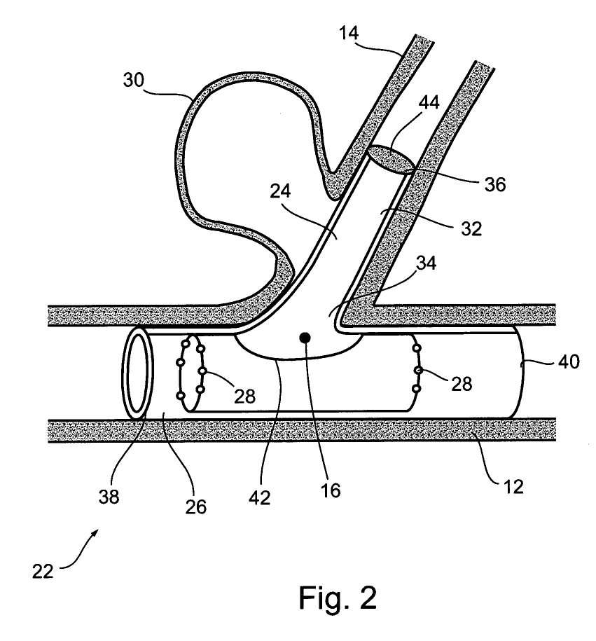

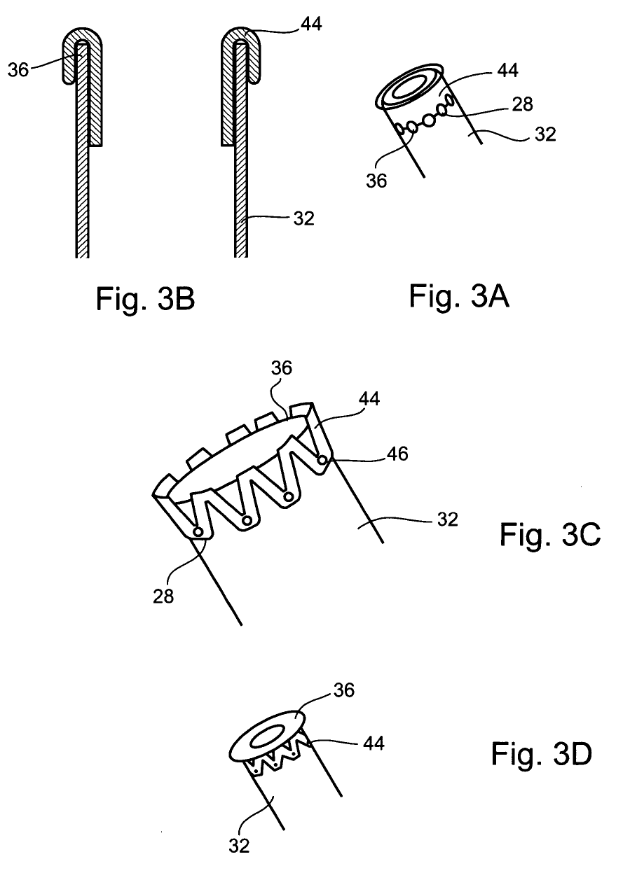

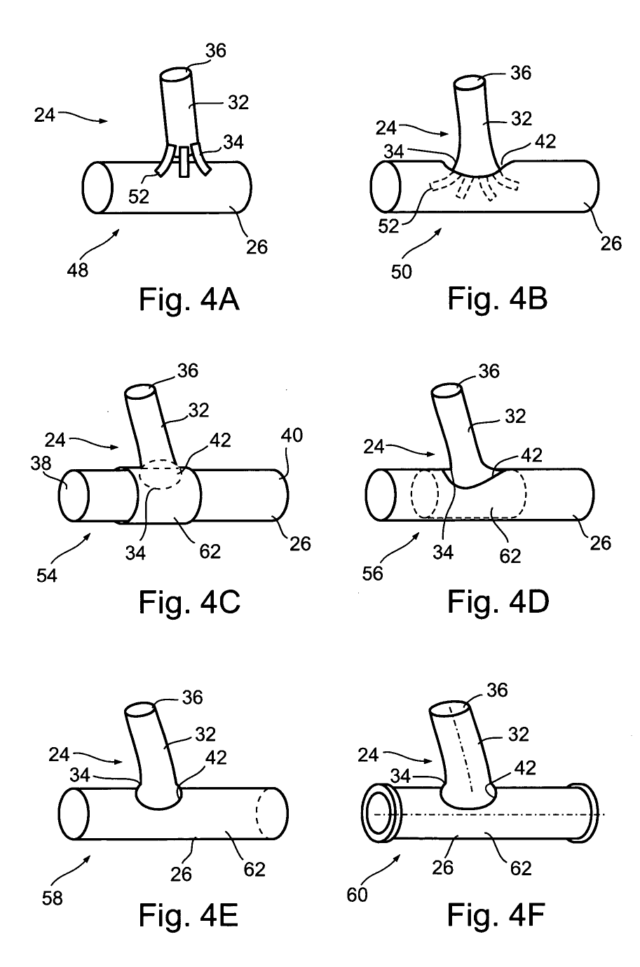

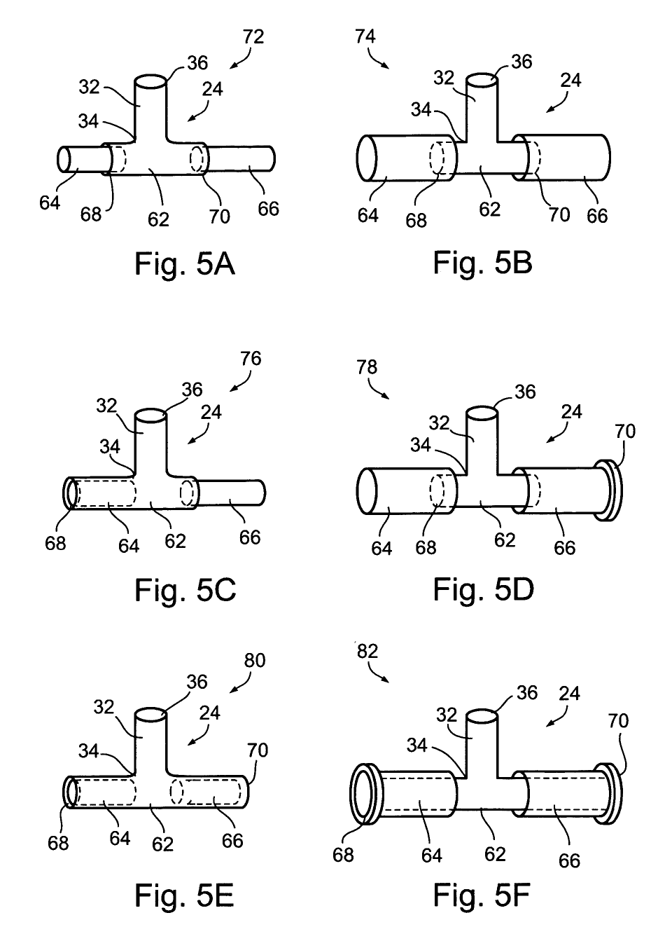

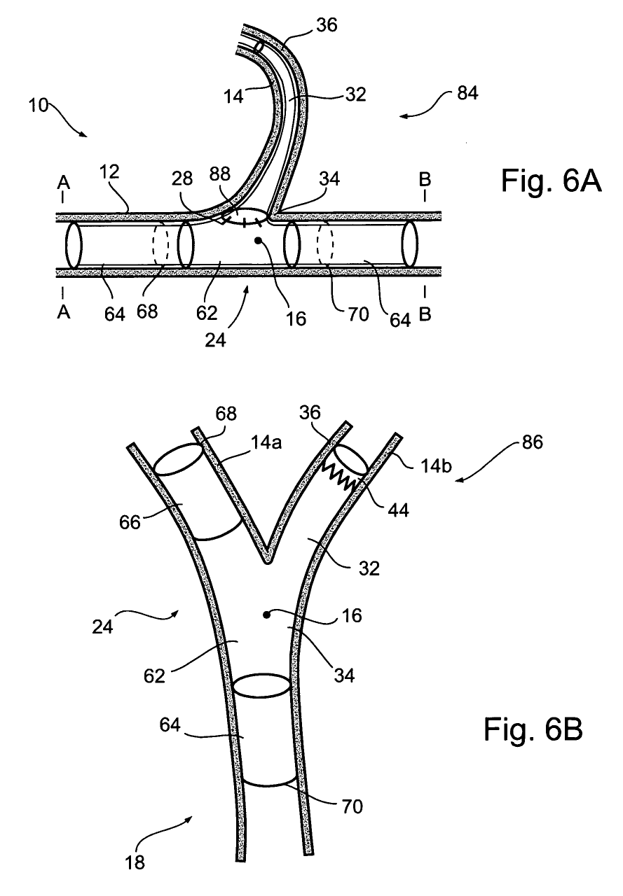

For convenience, different embodiments of the present invention will be discussed together with reference to common structural features. Embodiments of the graft-stent assembly of the present invention depicted in FIG. 2 and FIGS. 4A-4F include only one stent. Embodiments of the graft-stent assembly of the present invention depicted in FIGS. 5A-5F and FIGS. 6A-6B include two stents. Embodiments of the graft-stent assembly of the present invention depicted in FIG. 2, FIGS. 3A-3D and FIG. 6B are characterized as including an expandable ring-shaped support member to anchor a distal end of the tubular graft wall in place inside a branch vessel.

In FIG. 2 is depicted a graft-stent assembly 22 of the present invention comprising a graft 24 and a substantially tubular expandable stent 26. Graft 24 is secured to stent 26 by a plurality of sutures 28 (or optionally by adhesives, bending members, clamps, glue, hooks, piercing members, staples, other applicable mechanical mean or combinations thereof). Graft-stent assembly 22 is deployed in a bifurcated blood vessel (depicted in cross section) with a trunk vessel 12, a branch vessel 14 and a bifurcation point 16. On branch vessel 14 is an aneurism 30.

Graft 24 (e.g., made of serous tissue, such as from thinned heterologus pericardium) includes a substantially tubular graft wall 32 defining a graft bore, a proximal graft end 34 and a distal graft end 36. Stent 26 (e.g., any suitable prior art stent) has a first stent end 38, a second stent end 40 and a side opening 42. A part of graft 24 is found inside stent 26 so that tubular graft wall 32 emerges from side opening 42. In such a way, the graft bore and the stent bore are discontinuous, but are in fluid communication through proximal graft end 34. It is seen that in graft-stent assembly 22 the graft bore diverges from the stent bore by about 60°. In embodiments of the present invention, the graft bore diverges from a respective stent bore at an angle of greater than about 5°, greater than about 10° or even greater than about 30°. In FIG. 2, it is seen that the bore of graft 24 substantially defines a branch part of graft-stent assembly 22 and the bore of stent 26 defines a trunk part of graft-stent assembly 22.

In FIG. 2, stent 26 is in an expanded state within the bore of trunk vessel 12 and side opening 42 of stent 26 is located across bifurcation point 16, allowing tubular graft wall 32 to be located within the bore of branch vessel 14. Stent 26 supports patency of trunk vessel 12. Tubular graft wall 32 supports patency of bifurcation point 16 and of branch vessel 14, relines branch vessel 14, and provides a smooth transition from trunk vessel 12 into branch vessel 14, reducing turbulence in fluids flowing therethrough. Further, tubular graft wall 32 provides structural support to branch vessel 14 and bifurcation point 16 from radially outwards pressure caused by blood when the walls of branch vessel 14 are weakened, for example, by age, scarring or disease. Further, if branch vessel 14 or bifurcation point 16 crack or tear during of the deployment of graft-stent assembly 22, tubular graft wall 32 prevents leakage of blood and catastrophic failure of branch vessel 14. Further, tubular graft wall 32 is disposed across the entrance into aneurism 30 reducing the pressure inside aneurism 30 and preventing flow of fluids such as blood thereinto.

Inside the bore of tubular graft wall 32 and flush with distal graft end 36 is expandable ring-shaped support member 44. Ring-shaped support member 44 is secured to distal graft end 36 by adhesives (or optionally by sutures, bending members, clamps, glue, hooks, piercing members, staples, other applicable mechanical mean or combinations thereof). In graft-stent assembly 22 depicted in FIG. 2, ring-shaped support member 44 is found inside the bore of tubular graft wall 32 and flush with distal graft end 36 so that when deployed, ring-shaped support member 44 presses distal graft end 36 against the inner walls of branch vessel 14 securing distal graft end 36 in place inside branch vessel 14 without extending beyond distal graft end 36. In alternate embodiments, vide infra, support member 44 is located about graft member 32

In FIGS. 3A, 3B, 3C and 3D are depicted various embodiments of expandable ring-shaped support members 44 that are functionally associated with distal graft ends 36 and are configured to hold the respective distal graft ends 36 securely in place inside a branch vessel 14 when deployed therein.

Similarly to graft-stent assembly 22 depicted in FIG. 2, in the embodiments of the graft-stent assembly of the present invention depicted in FIGS. 3A and 3B, ring-shaped support member 44 is found inside the bore of tubular graft wall 32.

In the embodiment of the graft-stent assembly of the present invention depicted in FIG. 3A, ring-shaped support member 44 is found inside the bore of tubular graft wall 32 and distal graft end 36 (in phantom) is folded thereover so that ring-shaped support member 44 is encapsulated within a pocket defined by tubular graft wall 32 and distal graft end 36. The fold is secured by sutures 28. The perforations caused by sutures 28 in tubular graft wall 32 and the juncture where distal graft end 36 meets the inside of tubular graft wall 32 are sealed and smoothed by the application of glue, substantially as described in U.S. Pat. No. 6,579,307 to prevent leakage of and turbulence in fluids flowing through the bore of tubular graft wall 32. An advantage of such encapsulaton is that ring-shaped-support member 44 is securely associated with tubular graft 32 with no chance of incorrect placement, yet is not directly physically attached to graft 32. The lack of physcial attachment simplifies manufacture of such a graft-stent and prevents damage to tubular graft wall 32 during expansion of ring-shaped support member 44, for example tearing at sutures and the like.

In the embodiment of the graft-stent assembly of the present invention depicted in FIG. 3B (in a cross section), ring-shaped support member 44 is bent over and clamps distal graft end 36 of tubular graft wall 32. In such a way, ring-shaped support member 44 extends somewhat but not significantly beyond distal graft end 36. Suitable ring-shaped support members 44 configured to bend over and clamp a graft end are taught in are taught in PCT Patent Application No. IB01/00315 published as WO01/66037 of the inventor. An advantage of such configuration is that clamping holds distal graft end 36 securely at a plurality of locations about the circumference without perforating or otherwise damaging distal graft end 36.

Generally, in a graft-stent assembly of the present invention, an expandable ring-shaped support member 44 is secured to a respective distal graft end 36, preferably at a plurality of locations about the circumference of distal graft end 36, for example, using sutures (e.g., as depicted in FIG. 2), but also with adhesives, bending members, clamps, glue, hooks, piercing members, staples, laser welding other applicable mechanical mean or combinations thereof.

Unlike the embodiments of the graft-stent assembly of the present invention depicted in FIGS. 2, 3A and 3B, in the embodiments of the graft-stent assembly of the present invention depicted in FIGS. 3C and 3D, tubular graft wall 32 is threaded through a respective ring-shaped support member 44.

In the embodiment of the graft-stent assembly of the present invention depicted in FIG. 3C, distal graft end 36 is substantially flush with and does not significantly extend beyond ring-shaped support member 44 and is attached thereto with a plurality of sutures 28. A noteworthy feature depicted in FIG. 3C is that sutures 28 are threaded through a single row of eyelets 46 in ring-shaped support member 44, eyelets 46 all defining a circle about the central axis of tubular graft wall 32. Thus, when ring-shaped support member 44 of FIG. 3C expands during deployment, the radial distance between any two sutures 28 increases, but the distance in the axial direction remains unchanged. Such a disposition prevents uneven stress that may lead to tearing developing in tubular graft wall 32. A similar disposition of sutures is taught in PCT Patent Application No. IB01/00315 published as WO01/66037 of the inventor.

In an embodiment of the graft-stent assembly of the present invention depicted in FIG. 3D, distal graft end 36 is threaded through ring-shaped support member 44 and folded thereover.

In FIGS. 4A-4F are depicted embodiments of a graft-stent assembly of the present invention comprising a single stent 26 and a graft 24 having a tubular graft wall 32 with a distal graft end 36 and a proximal graft end 34. In the graft-stent assembly depicted in FIGS. 4A the bore of tubular graft wall 32 is in fluid communication with the bore of stent 26 through the wall of stent 26 through proximal graft end 34. In the graft-stent assemblies depicted in FIGS. 4B-4F stent 26 is provided with a side opening 42 and the bore of tubular graft wall 32 is in fluid communication with the bore of stent 26 through proximal graft end 34.

Like in graft-stent assembly 22 depicted in FIG. 2, in the graft-stent assemblies depicted in FIGS. 4A-4F the graft-bore diverges from a side of a respective stent 26. Like in graft-stent assembly 22, the graft bore substantially defines a branch implant of the respective graft-stent assembly and the bore of the stent substantially defines a trunk implant of the respective graft-stent assembly.

Unlike the graft-stent assemblies of the present invention depicted in FIGS. 2 and 3A-3D, the graft-stent assemblies depicted in FIGS. 4A-4F are not provided with an expandable ring-shaped support member associated with distal end 36 of a respective tubular graft wall 32. Rather, a graft 24 is anchored to a stent 26 and a respective tubular graft wall 32 is deployed loosely within a branch vessel 14. In such embodiments of a graft-stent of the present invention, a tubular graft wall 32 is deployed as-is in a branch vessel 14 with no further downstream anchoring. As the upstream end of tubular graft wall 32 is anchored to one or more stents, e.g., 26, the fluid (e.g., blood) flow holds tubular graft wall 32 extended and pressed against the inner surface of the bore of branch vessel 14 and keeps the graft bore open. One advantage is that deployment of such a graft-stent assembly is simple as there is no maneuvering and expansion of an expandable ring-shaped support member 44 required (vide infra). Another advantage is that fluid such as blood flowing through the branch vessel 14 holds tubular graft wall 32 tautly against the walls of the branch vessel 14, without wrinkles or creases and forces tubular graft walls 32 to conform to the shape of curved or tortuous branch vessels 14. When an expandable ring-shaped support member 44 is used to hold a distal graft end 36 in place, there is a possibility that the tubular graft wall 32 will be held taut and not be able to conform to the walls of a branch vessel 14.

A graft-stent assembly 48 depicted in FIG. 4A and a graft-stent assembly 50 depicted in FIG. 4B substantially comprise a stent 26 with a side opening 42 and a graft 24 including a tubular graft wall 32 having a distal graft end 36 and a proximal graft end 34. About the periphery of proximal graft end 34 are arrayed a plurality of connecting tabs 52. In graft-stent assembly 48 and graft-stent assembly connecting tabs 52 are strips of heterologous tissue (e.g., serous tissue, pleura, peritoneum, pericardium), but in other non-depicted embodiments are also bands, filaments, ribbons, strands, strings, strips or threads of any other suitable material.

Connecting tabs 52 are attached to stent 26 so as to anchor graft 24 to stent 26, for example using sutures, adhesives, bending members, clamps, glue, hooks, piercing members, staples, laser welding other applicable mechanical mean or combinations thereof, including simply being knotted about structural elements of stent 26. In graft-stent assembly 48 of FIG. 4A, tabs 52 are substantially disposed on the outside of stent 26 about the periphery of side opening 42 and contacting the outer surface of stent 26. In graft-stent assembly 50 of FIG. 4B, tabs 52 are substantially disposed on the inside of stent 26 about the periphery of side opening 42 and contacting the inner surface of stent 26, so that proximal end 34 of tubular graft wall 32 is pulled inwards into stent 26. Generally, a graft-stent assembly similar to assemblies 48 and 50 provided with connecting tabs 52 has at least one and preferably at least two tabs 52. In graft-stent assembly 48 of FIG. 4A, tabs 52 are secured to tubular graft wall 32, for example, using sutures, adhesives, bending members, clamps, glue, hooks, piercing members, staples, laser welding other applicable mechanical mean or combinations thereof. In graft-stent assembly 50 of FIG. 4B, connecting tabs 52 are integrally formed with tubular graft wall 32.

In preferred embodiments of the present invention, a graft 24 is provided with a second tubular graft part rather than connecting tabs 52 (or the equivalent) allowing better association with a respective stent 26. Better association includes such aspects as increased contact area between stent 26 and the second tubular graft part so as to distribute potentially deforming forces over a large area of a stent, better securing of graft 24 to individual stent 26, more accurate relative positioning of stent 26 with graft 24 and better sealing and support of a bifurcated vessel in which the graft-stent is deployed.

A graft-stent assembly 54 depicted in FIG. 4C, a graft-stent assembly 56 depicted in FIG. 4D, a graft-stent assembly 58 depicted in FIG. 4E and a graft-stent assembly 60 depicted in FIG. 4F, substantially comprise a stent 26 with a side opening 42 and a graft 24 including a tubular graft wall 32 having a distal graft end 36 and a proximal graft end 34 as well as a second substantially tubular graft part 62 having a bore not parallel to the graft bore of tubular graft wall 32 and configured to connect graft 24 to stent 26. As is seen in FIGS. 4C-4F, the bore of second tubular graft part 62 is substantially parallel to the bore of stent 26. Second tubular graft part 62 holds graft 24 so that proximal graft end 34 is in fluid communication with the bore of stent 26 through side opening 42.

In graft-stent assembly 54 depicted in FIG. 4C, second tubular graft part 62 surrounds a middle part of stent 26, where both first stent end 38 and second stent end 40 emerge from second tubular graft part 62.In graft stent assembly 58 depicted in FIG. 4E second tubular graft part 62 surrounds stent 26 in its entirety, substantially constituting an external stent jacket.

In graft stent assembly 56 depicted in FIG. 4D, second tubular graft part 62 is substantially entirely disposed within the bore of stent 26.

In graft-stent assembly 60 depicted in FIG. 4F, the middle part of second tubular graft part 62 is disposed within the bore of stent 26 while both the first end and the second ends of second tubular graft part 62 emerge from the ends of stent 26 and are folded thereover so that tubular graft part 62 substantially constitutes an internal stent jacket.

In FIGS. 5A-5F and FIGS. 6A-6B are depicted embodiments of a graft-stent assembly of the present invention comprising a first stent 64, a second stent 66 and a graft 24 having a tubular graft wall 32 with a distal graft end 36 and a proximal graft end 34, and a second tubular graft part 62 having a bore not parallel to the graft bore of tubular graft wall 32 and configured to connect graft 24 to first stent 64 and second stent 66. To this end, second tubular graft part 62 has a first end 68 that engages first stent 64 and a second end 70 that engages second stent 66. As is seen in FIGS. 5A-5F and FIGS. 6A-6B, the bore of second tubular graft part 62 is substantially parallel to the bore of first stent 64 and second stent 66 and substantially define a single fluid conduit which is in fluid communication with the bore of tubular graft wall 32 which is not parallel with and diverges therefrom. Analogously to previously discussed graft-stent assemblies of the present invention, for graft-stent assemblies 72, 74, 76, 78, 80, 82 and 84 (depicted in FIGS. 5A-5F and 6A, respectively) the graft bore substantially defines a branch implant of the respective graft-stent assembly and the bores of first stent 64, second stent 66 and the second tubular graft part 62 substantially defines a trunk implant of the respective graft-stent assembly (see FIG. 6A). Stent assembly 86 depicted in FIG. 6B is somewhat different and will be discussed hereinbelow.

An advantage of a graft-stent assembly of the present invention including two independent stents that are physically associated only by a second tubular graft part is increased flexibility and a total lack of mechanical coupling between the two stents. Since first stent 64 and second stent 66 are devoid of mutual physical association or contact and attached only through second tubular graft part 62 of graft 24, manipulation and expansion of any one stent during deployment of a respective graft-stent assembly does not cause any deformation such as buckling, bending or stretching in the other stents. Further, the need for a side opening in a stent is obviated. Further, any two stents having different properties (e.g., having different expanded or unexpanded radii, different compression and expansion resistance, having different geometries and construction, of different materials) may be coupled in one graft-stent assembly. The importance of coupling two stents having different properties is discussed below with reference to FIGS. 6A and 6B.

Unlike the graft-stent assemblies depicted in FIGS. 2 and 3A-3D and like the graft-stent assemblies depicted in FIGS. 4A-4F, the graft-stent assemblies depicted in FIGS. 5A-5F are not provided with an expandable ring-shaped support member 44 associated with the distal end of a respective tubular graft wall 32.

In graft-stent assembly 72 depicted in FIG. 5A, first end 68 of second tubular graft part 62 surrounds an end of first stent 64 so that an end of first stent 64 emerges from first end 68 and second end 70 of second tubular graft part 62 surrounds an end of second stent 66 so that an end of second stent 66 emerges from second end 70.

In graft-stent assembly 74 depicted in FIG. 5B, first end 68 of second tubular graft part 62 is threaded into the bore of first stent 64 through an end of first stent 64 and is disposed within the bore of first stent 64 and second end 70 of second tubular graft part 62 surrounds an end of second stent 66.

In graft-stent assembly 76 depicted in FIG. 5C, first stent 64 is entirely surrounded by first end 68 of second tubular graft part 62 and second end 70 of second tubular graft part 62 surrounds an end of second stent 66 so that an end of second stent 66 emerges from second end 70. Second tubular graft part 62 substantially constitutes an external stent jacket of first stent 64.

In graft-stent assembly 78 depicted in FIG. 5D, first end 68 of second tubular graft part 62 is threaded into the bore of first stent 64 through an end of first stent 64 and is disposed within the bore of first stent 64 and second end 70 of second tubular graft part 62 is threaded through the bore of second stent 66 through a first end of second stent 66 and is disposed within the bore of second stent 64, emerging through a second end of second stent 66 to be folded thereover. Second tubular graft part 62 substantially constitutes an internal stent jacket of second stent 66.

In graft stent assembly 80 depicted in FIG. 5E, first stent 64 is entirely surrounded by first end 68 of second tubular graft part 62 and second stent 66 is entirely surrounded by second end 68 of second tubular graft part 62. Second tubular graft part 62 substantially constitutes an external stent jacket of first stent 64 and of second stent 66.

In graft stent assembly 82 depicted in FIG. 5F, first end 68 of second tubular graft part 62 is threaded through the bore of first stent 64 through a first end of first stent 64 and is disposed within the bore of first stent 64, emerging through a second end of first stent 64 to be folded thereover and second end 70 of second tubular graft part 62 is threaded through the bore of second stent 66 through a first end of second stent 66 and is disposed within the bore of first stent 66, emerging through a second end of second stent 66 to be folded thereover. Second tubular graft part 62 substantially constitutes an internal stent jacket of first stent 64 and of second stent 66.

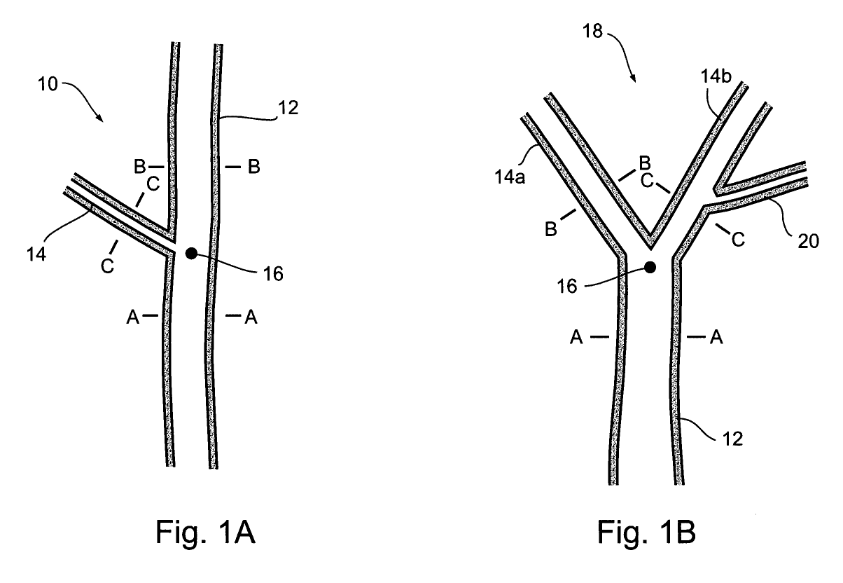

Graft-stent assembly 84 depicted in FIG. 6A is substantially similar to graft-stent assembly 72 depicted in FIG. 5A. In FIG. 6A graft-stent assembly 84 includes two independent stents 64 and 66 deployed in a bifurcated object 10 (e.g., an artery) such as depicted in FIG. 1A, with a small bore branch vessel 14 and a trunk vessel 12 where trunk vessel 12 has a larger bore upstream of bifurcation point 16 than downstream of bifurcation point 16. Tubular graft wall 32 of graft-stent assembly 84 is deployed in a branch vessel 14, first stent 64 is deployed inside trunk vessel 12 upstream of bifurcation point 16 and second stent 66 is deployed inside trunk vessel 12 downstream of bifurcation point 16. As first stent 64 and second stent 66 are not mechanically coupled, first stent 64 and second stent 66 are each expanded to a different size appropriate to support the respective part of trunk vessel 12 without causing excessive mechanical stress to bifurcation point 16 or to the smaller (downstream) part of trunk vessel 12.

Graft-stent assembly 86 depicted in FIG. 6B is substantially similar to graft-stent assembly 80 depicted in FIG. 5E in that both first stent 64 and second stent 66 are substantially entirely surrounded by second tubular graft part 62 of graft-stent assembly 86, with at least one notable difference that graft-stent assembly 84 is provided with an expandable ring-shaped support member 44. In FIG. 6B graft-stent assembly 86 is deployed in a bifurcated object 18 (e.g., an artery) such as depicted in FIG. 1B where neither of the two branch vessels 14A or 14B is substantially smaller than the other. In such a case, it can be said that the bore of first stent 64 together with the bore of a part of second tubular graft part 62 that is proximate to first end 68 substantially defines a trunk implant of graft-stent assembly 86, that the bore of second stent 66 together with the bore of a part of second tubular graft part 62 that is proximate to second end 70 substantially defines a first branch implant of graft-stent assembly 86, and that tubular graft wall 32 together with ring-shaped support member 44 substantially defines a second branch implant of graft-stent assembly 86.

A graft-stent assembly of the present invention is made of a number of components, at the very least one stent and a graft 24 including a tubular graft wall 32. Generally but not necessarily a graft is secured to a stent or stents using sutures, adhesives, bending members, clamps, glue, hooks, piercing members, staples, laser welding other applicable mechanical mean or combinations thereof.

As discussed above, second tubular graft parts 62 of graft-stent assemblies of the present invention are disposed either on the outside of a respective stent or inside the bore of a respective stent. Advantages of disposing a second tubular graft part 62 inside the bore of a respective stent include providing a smooth lumen allowing unrestricted and non-turbulent flow of fluids through the graft-stent assembly. Further, such an arrangement is useful for deployment in weakened or damaged vessels as part of the force applied by fluids flowing through such a graft-stent assembly is dissipated by the stent and not transferred to the vessel.

Advantages of disposing a second tubular graft part 62 on the outside of a respective stent include secure and even expansion of second tubular graft parts 62. Further, although a second tubular graft parts 62 may be attached to a stent using, for example, sutures, adhesives, bending members, clamps, glue, hooks, piercing members, staples, laser welding other applicable mechanical mean or combinations thereof, a second tubular graft part 62 placed about the outside of a respective stent often allows avoiding the use of sutures or other methods of securing the graft to the stents as the second tubular graft part is held in place by tension and the attendant friction. Further, in two-stent embodiments of a graft-stent assembly, such an arrangement encases the area where any two stents meet, preventing pinching of bodily parts between the stents.

As noted hereinabove, it is preferable that the material from which a graft of the present invention is fashioned be flexible. In embodiments of the present invention it is also preferable that the graft be substantially elastic so as to better retain a desired shape during manipulation and deployment.

In the art stents with a jacket made of a material that is porous and permeable so as to allow cells to grow into and through the material, ultimately leading to the jacket becoming an integral part of the vessel in which deployed are known, see for example U.S. Pat. No. 5,723,004. In embodiments of the present invention, the material from which a graft is fashioned is porous and/or permeable so as to allow tissue in-growth. A disadvantage of such permeability is that smooth muscle cells are known to grow in a disorganized and ultimately restenotic fashion through porous and permeable membranes.

In the art stents with a jacket made of a material that is substantially impervious to tissue proliferation therethrough are known, see for example PCT Patent Application PCT/IB98/01459 published as WO 99/15105 of the inventor. Such imperviousness is useful in preventing tissue buildup on and through the material and prevents the migration of smooth muscle cells. In embodiments of the present invention, the material from which a graft is fashioned is substantially impervious to tissue proliferation therethrough. Such embodiments are useful for providing a treated vessel with a smooth lining that is substantially impervious to restenosis.

In the art stents with a jacket made of a material that is impermeable to fluids so as to form a sealed vessel and thus avoid extravasation of fluids through the material are known. In embodiments of the present invention, the material from which a graft is made is impermeable to fluids.

Useful materials from which to fashion a graft of the present invention include synthetic or polymeric material including but not limited to polytetrafluoroethylene, urethane, elastomer, polyamide (e.g., Nylon) and polyester (e.g., Dacron).

Useful materials from which to fashion a graft of the present invention are also biological tissue including but not limited to autologous tissue, heterologous tissue, venous tissue, arterial tissue, serous tissue, pleura, peritoneum, pericardium and aortic leaflet. Generally suitable tissue types include but are not limited to equine, porcine, bovine or human tissue. It is often preferred that the tissue be thinned, that is after harvesting one or more layers of the harvested tissue are removed, e.g. by scraping, shaving, slicing or skiving (see U.S. Pat. Nos. 6,468,300 and 6,254,627 of the inventor). In order to increase the toughness of the tissue, it is often advantageous to treat the tissue, for example with a glutaraldehyde or a phosphate solution, in order to cross-link collagen in the tissue.

Serous membranes are made of two strata of tissue. The serous stratum or layer of a serous membrane is a very smooth single layer of flattened, nucleated mesothelial cells united at their edges by a cement substance. The serous cells rest on a basement layer or stratum, a rough, strong fibrous layer. Serous membrane is one material that is strong, elastic and thin enough to be useful in implementing the teachings of the present invention. Thinned serous membrane is even more preferred as noted above and as taught in U.S. Pat. Nos. 6,254,627 and 6,468,300 of the inventor. Not only is thinned serous membrane sufficiently strong, elastic and even thinner than serous membrane, thinned serous membrane also provides little resistance to radial expansion, making thinned serous membrane one of the few materials suitable for use in covering or jacketing self-expanding stents. Thus, in a preferred embodiment, the graft is substantially fashioned from a thinned serous membrane where a harvested serous membrane (peritoneum, pericardium or pleural tissue especially porcine, bovine, equine and human serous tissue) has been processed by removal of a layer of at least some of the basement tissue layer (and thus thinned), preferably removal of all the basement tissue, leaving only the serous tissue layer. In an embodiment of the present invention only a part of the basement tissue layer is removed. In an embodiment of the present invention, the thinned serous membrane is substantially serous tissue devoid of basement tissue. In an embodiment of the present invention, the material consists essentially of serous tissue.

When serous membrane or thinned serous membrane is used to implement the teachings of the present invention it is usually preferred to orient the membrane so that the smooth serous strata is facing the fluid flow to reduce turbulent flow.

A graft of a graft-stent assembly of the present invention is fashioned from one or more parts according to any of the methods with which one skilled in the art is acquainted.

In an embodiment of the present invention, tubular parts of a given graft are fashioned from a planar or substantially planar sheet of an appropriate membrane (synthetic or harvested), generally by rolling the sheet (with abutting or overlapping edges) and then fixing the tubular shape using any of the methods known in the art including but not limited to sutures, adhesives, bending members, clamps, glue, hooks, piercing members, staples, other applicable mechanical mean or combinations thereof). A graft made up of more than one tubular part is conveniently fashioned by making two separate tubes and conjoining the two tubes (using, for example, sutures, adhesives, bending members, clamps, glue, hooks, piercing members, staples, other applicable mechanical mean or combinations thereof).

In an embodiment of the present invention a graft is fashioned including seamless tubular parts. In an embodiment, a seamless tubular part of a graft of the present invention is fashioned from synthetic materials by methods including but not limited to, weaving synthetic fibers, molding a polymer or welding the seam of a rolled sheet, for instance using heat or an appropriate solvent. In an embodiment, a seamless tubular part of a graft of the present invention is fashioned from a section of harvested tubular biological tissue. In such a case a suitable tubular vessel, such as an appropriately sized autologous, homologous or heterologous artery or vein is identified, harvested, isolated and treated to prepare a seamless tubular graft component. Treatments include chemical or biological treatments, for example cross-linking or digestion, to increase flexibility or strength of the material of the tubular graft component. Treatments also include mechanical thinning to increase the flexibility and reduce thickness of the material of the graft component.

In embodiments of the present invention, a graft is made of two different materials. For example, the tubular graft wall is fashioned from a thin tube of serous tissue attached to a second tubular graft part made of a thicker material, for example, a harvested artery.

In an embodiment of the present invention a bifurcated graft is seamless. In an embodiment, a seamless bifurcated graft of the present invention is fashioned from synthetic materials by methods including but not limited to, weaving synthetic fibers, molding a polymer or welding separate components, for instance using heat or an appropriate solvent. In a preferred embodiment, a seamless bifurcated graft of the present invention is fashioned from a section of a bifurcated biological tissue. In such a case a suitable bifurcated vessel, such as an appropriately sized autologous, homologous or heterologous bifurcated artery or vein is identified, harvested, isolated and treated to prepare a seamless bifurcated graft. Treatments include chemical or biological treatments, for example cross-linking or digestion, to increase flexibility or strength of the material of the vessel and ultimately of the thus-fashioned graft. Treatments also include mechanical thinning to increase the flexibility and reduce thickness of the material of the vessel.

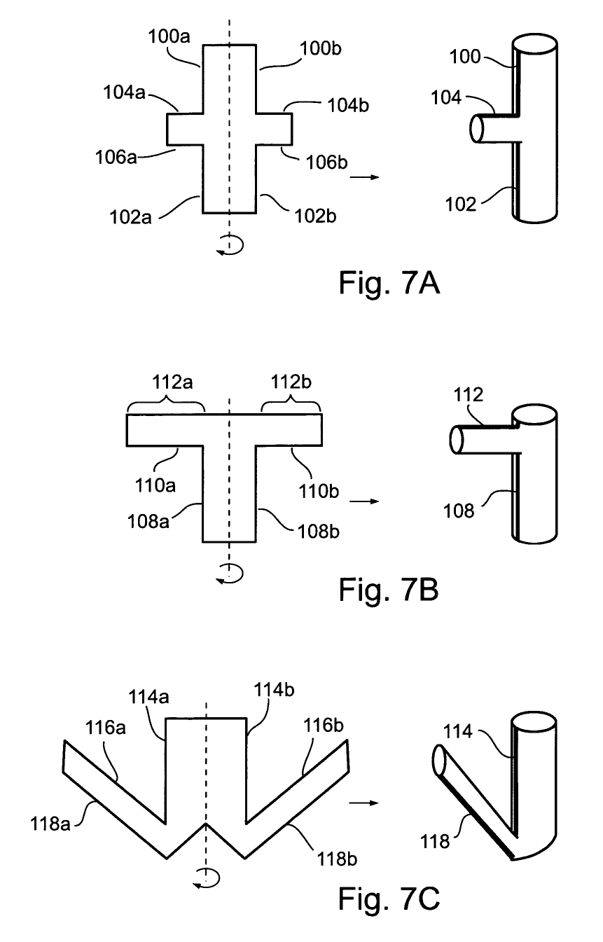

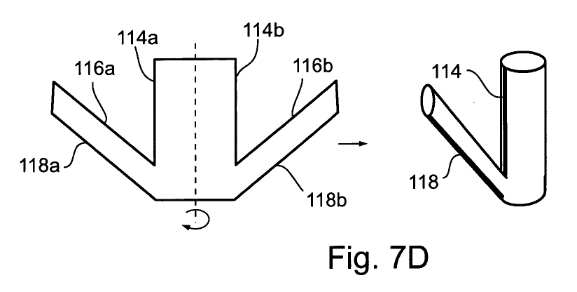

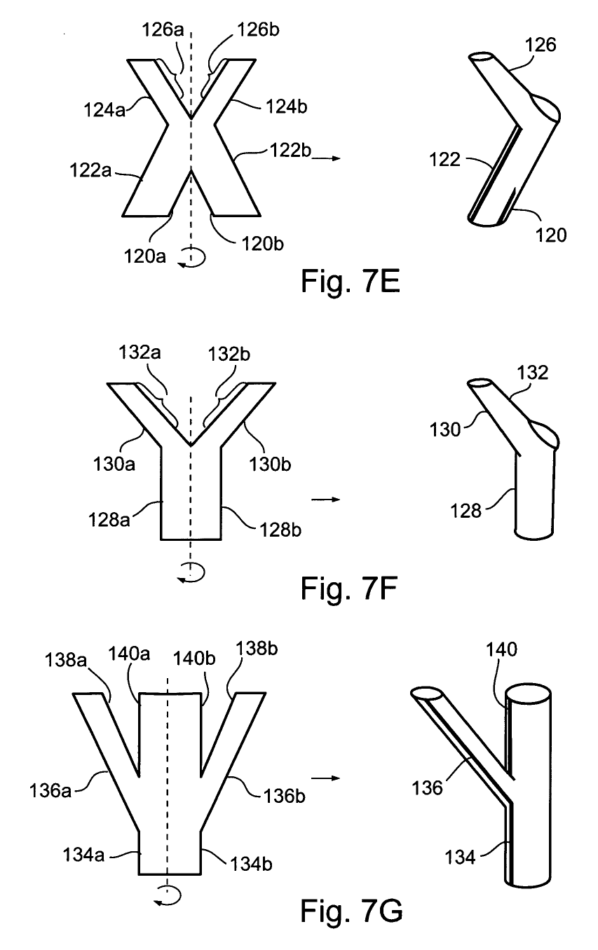

In an embodiment of the present invention, a bifurcated graft of the present invention is essentially fashioned from one sheet of planar or substantially planar sheet of an appropriate membrane (synthetic or harvested), rolled up into shape (with abutting or overlapping edges) and then fixed in shape using any of the methods known in the art including but not limited to sutures, adhesives, bending members, clamps, glue, hooks, piercing members, staples, other applicable mechanical mean or combinations thereof). Such an embodiment is preferred over a seamless bifurcated graft as the availability of suitable bifurcated vessels for harvesting is limited and limits the materials from which grafts are fashioned. Such an embodiment is preferred over a bifurcated graft fashioned from two or more conjoined tubular components due to the reduced number of seams. Preferred shapes of sheets suitable for use in fashioning a bifurcated graft from one sheet of material generally include shapes having one C2 symmetry axis and at least one external angle of no greater than 90° and/or at least 7sides. In FIGS. 7A-7G are depicted various suitable shapes rolled up and edges attached to fashion bifurcated grafts of the present invention.

In FIG. 7A is depicted a cross-shaped sheet, rolled up as depicted by the arrow so that sides 100a and l00b are joined to form seam 100, sides 102a and 102b are joined to form seam 102, sides 104a and 104b are joined to form seam 104 and sides 106a and 106b are joined to form a non-depicted seam to fashion a bifurcated graft.

In FIG. 7B is depicted a “T”-shaped sheet, rolled up as depicted by the arrow so that sides 108a and 108b are joined to form seam 108, sides 110a and 110b are joined to form a non-depicted seam and sides 112a and 112b (delimited by the braces) are joined to form a seam 112 to fashion a bifurcated graft.

In FIGS. 7C and 7D are depicted “W”-shaped sheets, rolled up as depicted by the arrow so that sides 114a and 114b are joined to form seam 114, sides 116a and 116b are joined to form a non-depicted seam and sides 118a and 118b are joined to form a seam 118 to fashion a bifurcated graft.

In FIG. 7E is depicted an “X”-shaped sheet, rolled up as depicted by the arrow so that sides 120a and 120b are joined to form seam 120, sides 122a and 122b are joined to form seam 122, sides 124a and 124b are joined to form a non-depicted seam, and sides 126a and 126b (delimited by the braces) are joined to form a seam 126 to fashion a bifurcated graft.

In FIG. 7F is depicted a “Y”-shaped sheet, rolled up as depicted by the arrow so that sides 128a and 128b are joined to form seam 128, sides 130a and 130b are joined to form seam 130 and sides 132a and 132b are joined to form seam 132 to fashion a bifurcated graft.

In FIG. 7G is depicted a “105 ”-shaped sheet, rolled up as depicted by the arrow so that sides 134a and 134b are joined to form seam 134, sides 136a and 136b are joined to form seam 136, sides 138a and 138b are joined to form a non-depicted seam and sides 140a and 140b are joined to form seam 140 to fashion a bifurcated graft.

Any suitable method known in the art may be used to conjoin any two components or to attach ends of a single piece of material to fashion a graft of the present invention. Such methods include but are not limited to sutures, adhesives, clamps, glue, hooks, piercing members, staples, laser welding other applicable mechanical mean or combinations thereof. In embodiments of the present invention two components or two ends of a single piece of material are overlapped and joined. An overlapping joint is leak resistant and strong due to increased surface area of the seam. In embodiments of the present invention two components or two ends of a single piece of material are abutted and joined. An abutting joint is thinner than an overlapping joint.

Once a graft of the present invention of the desired topography is made, assembly of a graft-stent assembly is straightforward for one skilled in the art upon perusal of the disclosure herein.

In one-stent embodiments of a graft-stent of the present invention (e.g., as depicted in FIG. 2, or FIGS. 4A-4F), a suitable stent is selected and contacted with an appropriate part of the graft. When a stent is surrounded by a second tubular part of a graft, the stent is generally threaded through the second tubular part to the desired extent, e.g., so that the tubular graft wall is across a side opening of the stent. Preferably a stent is secured to a graft using a suitable method, including but not limited to the use of sutures, adhesives, bending members, clamps, glue, hooks, piercing members, staples, laser welding other applicable mechanical mean or combinations thereof and/or by a combination of tension and concomitant friction of a second tubular graft part surrounding a stent. In two-stent embodiments of a graft-stent of the present invention, two suitable stents (identical or different) are selected, contacted with and, if desired, attached to the second tubular graft part substantially as described above.

In embodiments of a graft-stent of the present invention including an expandable ring-shaped support member a suitable expandable ring-shaped support member is selected, contacted with and secured to an appropriate part of the graft. One skilled in the art is acquainted with suitable expandable ring-shaped support members, for example expandable ring-shaped support members as described in PCT Patent Application No. IB01/00315 published as WO01/66037 of the inventor.

One of the design features of embodiments of a graft of the present invention comprising a second tubular part (e.g., embodiments including those depicted in FIGS. 2, 4C-4F, 5A-5F and 6A-6B) is the length of the second tubular part relative to a stent with which the second tubular part is associated, that is to say, what part of the outer surface of a stent is covered by a respective second tubular part (when the second tubular part is disposed outside the stent) or what part of the inside surface of a stent is in contact with a respective second tubular part (when the second tubular part is disposed inside the stent).

In embodiments of a one-stent graft-stent assembly of the present invention a second tubular part is substantially as long or even longer than a stent with which the second tubular part is associated.

In embodiments of a two-stent graft-stent assembly of the present invention a second tubular part is substantially as long or even longer than both stents with which the second tubular part is associated.

In embodiments of the present invention, a second tubular part is shorter than an associated stent, being up to 90% of the length of an associated stent, being up to 70% of the length of an associated stent, being up to 50% of the length of an associated stent, being up to 30% of the length of an associated stent or even being up to 20% of the length of an associated stent.

It is known in the art to deploy a stent provided with a stent jacket. The stent jacket is generally a tubular membrane placed on the outer surface of the stent although internal stent jackets, substantially tubular membranes held within the bore of the stent, are known (see, for example, U.S. Pat. Nos. 6,254,627 and 6,699,277). The stent jacket provides a smooth lumen for the treated vessel, reduces turbulent flow through the treated vessel, and provides structural reinforcement. In embodiments of the present invention, one or more of the component stents of a graft-stent assembly are jacketed. For example, any of the different stent jackets known to one skilled in the art are useful for jacketing one, some or all component stents of a graft-stent assembly of the present invention.

It is known in the art to deploy a coated stent. Many different coatings are known in the art, for example, anti-thrombogenic coatings, anti-angiogenic coatings, anti-coagulant coatings and active pharmaceutical ingredient delivering coatings. Any of the different stent coatings known to one skilled in the art are useful for coating one, some or all component stents of a graft-stent assembly of the present invention.

Generally the walls of a graft of a graft-stent assembly of the present invention are as thin as possible yet are elastic enough and strong enough to be useful, that is to allow navigation and deployment of the graft-stent assembly including movement of the component stents of the graft-stent assembly without tearing. Clearly, the nature of the material from which a given graft is made determines in part the thickness of that graft. That said, a graft of a graft-stent assembly of the present invention preferably has walls that are not thicker than about 0.75 mm, not thicker than about 0.45 mm, not thicker than about 0.25 mm, not thicker than about 0.20 mm, and even not thicker than about 0.05 mm. A useful material from which to fashion a graft of a graft-stent assembly of the present invention having the appropriate thickness yet also being smooth to reduce the chances of restenosis, being sufficiently strong and flexible is thinned serous membrane as discussed above, especially bovine pericardium as disclosed in PCT Patent application No. PCT/IB98/01459 published as WO 99/15105 of the inventor.

Generally, there are no limitations on the outer diameter of a tubular graft wall of a graft-stent assembly of the present invention that may be relatively large or relatively small, depending on the diameter of the branch vessel in which the assembly is to be deployed. For example, a tubular graft wall configured for deployment in the peripheral cardiovascular system is generally of a relatively large outer diameter, for example greater than about 5 mm and typically greater than about 10 mm. That said, an advantage of the present invention is that the tubular graft wall may be of a relatively small diameter especially suitable for deployment in the coronary or the cerebrovascular systems. In embodiments of the present invention, a tubular graft wall is provided having an outer diameter not greater than about 6 mm, 5 mm, 4 mm, 3 mm, 2 mm and even not greater than 1 mm.

Generally, the length of the substantially tubular graft wall of a graft-stent assembly of the present invention is decided by a health care professional in accordance with the site of deployment, the geometry of the branch vessel in which the tubular graft wall is deployed and the state of the branch vessel.

It is clear to one skilled in the art upon perusal of the description herein, that it is possible to supply a graft-stent assembly of the present invention with an exceptionally long tubular graft wall and trim the graft wall in accordance with an immediate medical need. Thus in an embodiment of the present invention, a tubular graft wall is provided having a length greater than 20 mm, 40 mm, 60 mm, 80 mm and even greater than 100 mm.

In embodiments of the present invention, the tubular graft wall is provided having a ready-to-use length. Thus, in embodiments of a graft-stent assembly of the present invention a tubular graft wall has a length of up to about 60 mm, up to about 40 mm, or even up to about 25 mm. In embodiments of a graft-stent assembly of the present invention a tubular graft wall has a length of greater than about 1 mm, greater than about 2 mm, or even greater than about 3 mm. Generally, the tubular graft wall of a graft-stent assembly of the present invention is between about 5 mm and about 25 mm in length.

Generally, the outer diameter of a tubular graft wall is selected to be as close as possible to the size of the bore of the branch vessel in which the graft is to be deployed. In embodiments of the present invention, the graft is made of a stretchable material having an outer diameter equal to or slightly smaller than the inner diameter of the bore of the branch vessel. In such cases, the flow of fluid (e.g., blood) in the branch vessel pushes the graft against the inner wall of the branch vessel. In embodiments of the present invention, the graft has an outer diameter equal to or slightly larger than the inner diameter of the bore of the branch vessel. That said, in embodiments of a graft-stent assembly of the present invention a substantially tubular graft wall an outer diameter of at least about 0.5 mm, at least about 1 mm, at least about 2 mm, at least about 5 mm, and even at least about 10 mm.

Generally any type of stent known in the art is useful as a component of a graft-stent assembly of the present invention. Such stents include but are not limited to stents marketed by affiliates (e.g., Cordis, Centocor) of Johnson & Johnson, Guidant (Indianapolis, Ind., USA), Medtronic (Minneapolis, Minn., USA), Medinol (Tel Aviv, Israel), Cook Inc. (Bloomington, Ind., USA) and PM Devices Inc. (Richmond, British Columbia, Canada). In embodiments of the graft-stent assembly of the present invention, a first expandable stent and a second expandable stent (if present) are of substantially similar or identical dimensions, especially length, expanded diameter and/or unexpanded diameter.

In embodiments of the present invention a first expandable stent and a second expandable stent (if present) are of substantially different dimensions, especially length, expanded diameter and/or unexpanded diameter. In cases where the first expandable stent and a second expandable stent are of different dimensions, generally the first expandable stent is larger (especially of larger expanded and/or unexpanded diameter) as it is generally the first expandable stent that is destined to be deployed in a larger bored upstream part of a trunk vessel.

In embodiments of the present invention, the diameter (expanded or unexpanded) of the first expandable stent is substantially similar to the respective diameter of a second expandable stent.

In embodiments of the present invention, the diameter (expanded or unexpanded) of the first expandable stent is larger than the respective diameter of a second expandable stent.

In embodiments of a graft-stent assembly of the present invention an expandable stent has a length of up to about 80 mm, up to about 65 mm, or even up to about 50 mm.

In embodiments of a graft-stent assembly of the present invention an expandable stent has a length of greater than about 5 mm, greater than about 7 mm, or even greater than about 10 mm.

In embodiments of a graft-stent assembly of the present invention a second expandable stent (if present) has a length of up to about 80 mm, up to about 65 mm, or even up to about 50 mm.

In embodiments of a graft-stent assembly of the present invention a second expandable stent (if present) has a length of greater than about 5 mm, greater than about 7 mm, or even greater than about 10 mm.

Two important parameters used when selecting a stent for use are the expanded and unexpanded diameters of the stent.

Generally it is important that the unexpanded diameter of a stent be as small as possible to ease navigation through the bodily lumen to the deployment location. That said, the unexpanded diameter must be large enough to allow threading of the stent onto a deployment catheter and, if necessary, a stent-expanding device such as a stent-expanding balloon. Although there may be some variation in the unexpanded diameter of even two identical stents depending on how the two stents are used, herein by unexpanded diameter is intended the outer diameter of an expandable stent when crimped to the greatest extent onto a delivery catheter for deployment.

In embodiments of a graft-stent assembly of the present invention, a first expandable stent has an unexpanded diameter as defined above of at least about 0.5 mm, at least about 1 mm and even at least about 2 mm and a second expandable stent (if present) has an unexpanded diameter as defined above of at least about 0.5 mm, at least about 1 mm, and even at least about 2 mm. Specifically, in embodiments of a graft-stent assembly of the present invention configured for deployment in the coronary or cerebrovascular systems- a first expandable stent and/or a second expandable stent (if present) each has an unexpanded diameter of between about 0.5 mm and about 3 mm. In embodiments of a graft-stent assembly of the present invention configured for deployment in the peripheral vascular systems a first expandable stent and/or a second expandable stent (if present) each has an unexpanded diameter of between about 2.5 mm and about 6 mm.

Generally, any given stent has a wide range of expanded diameters. The expanded diameter of a stent subsequent to deployment is determined by the user of the stent according to medical criteria including the natural size of the lumen of the vessel in which the stent is deployed. Self-expanding stents are characterized by a specific maximal expansion that is the maximal diameter of the stent when the stent is free from externally applied forces. Most not self-expanding stents are also characterized by a maximal expansion that is the greatest extent to which the stent is expandable without comprising the structural integrity thereof.

In embodiments of a graft-stent assembly of the present invention a first expandable stent has a maximal expanded diameter as defmed above of up to about 30 mm, up to about 8 mm, up to about 6 mm, and even up to about 5 mm.

In embodiments of a graft-stent assembly of the present invention a second expandable stent (if present) has a maximal expanded diameter as defined above of up to about 30 mm up, to about 8 mm, up to about 6 mm, and even up to about 5 mm.

Generally any type of expandable ring-shaped support member known in the art is useful as a component of a graft-stent assembly of the present invention. Both self-expanding and not self-expanding expandable ring-shaped support members are suitable for implementing the teachings of the present invention. Exceptionally useful rings are those described in PCT Patent Application No. IB01/00315 published as WO01/66037 of the inventor. Also useful, for example, are expandable ring-shaped support members that are substantially one of the ring sections making up a stent such as described in U.S. Pat. No. 6,699,277 of the inventor.

As an expandable ring-shaped support member acts to anchor an end of a tubular graft wall in place and is not generally configured to support a vessel in which deployed, the axial length of an expandable ring-shaped support member is small, generally substantially smaller than of a stent. In embodiments of a graft-stent assembly of the present invention, an expandable ring-shaped support member has a length of less than about 10 mm, less than about 7 mm, less than about 5 mm, less than about 3 mm and even less than about 1 mm. In embodiments of the graft-stent assembly of the present invention, the axial length of an expandable ring-shaped support member is substantially shorter than the axial length of a respective graft wall. In embodiments of the present invention, the axial length of ring-shaped support member is less than about 30% and even less than about 10% of the axial length of respective graft wall.

Two important parameters used when selecting an expandable ring-shaped support member for use in implementing the teachings of the present invention are the expanded and unexpanded diameters of the expandable ring-shaped support member. Generally it is important that the unexpanded diameter of an expandable ring-shaped support member be as small as possible to ease navigation through the bodily lumen to the deployment location. That said, the unexpanded diameter must be large enough to allow threading of the expandable ring-shaped support member onto a deployment catheter and, if necessary, a ring-expanding device such as a stent-expanding balloon. Although there may be some variation in the unexpanded diameter of even two identical rings depending on how the two rings are used, herein by unexpanded diameter is intended the outer diameter of an expandable ring-shaped support member when crimped to the greatest extent onto a delivery catheter for deployment.

In embodiments of a graft-stent assembly of the present invention an expandable ring-shaped support member has an unexpanded diameter as defined above of at least about 0.5 mm, at least about 1 mm, and even at least about 2 mm.

Generally, any given expandable ring-shaped support member has a wide range of expanded diameters larger than a respective unexpanded diameter. The expanded diameter of an expandable ring-shaped support member subsequent to deployment is determined by the user of the graft-stent assembly according to medical criteria including the natural size of the lumen of the vessel in which the expandable ring-shaped support member is deployed. That said, self-expanding ring-shaped support member are characterized by a specific maximal expansion that is the maximal diameter of the ring-shaped support member when the ring-shaped support member is free from externally applied forces. Most not self-expanding ring-shaped support member are also characterized by a maximal expansion that is the greatest extent to which the ring-shaped support member is expandable without comprising the structural integrity thereof.

In embodiments of a graft-stent assembly of the present invention a expandable ring-shaped support member has a maximal expanded diameter as defined above of up to about 30 mm, up to about 8 mm, up to about 6 mm, and-even up to about 5 mm.

Expandable ring-shaped support members suitable for use in implementing the teachings of the present invention are taught in PCT Patent Application No. IB01/00315 published as WO01/66037 of the inventor. Expandable ring-shaped support members suitable for implementing the teachings of the present invention may be self-expanding or non self-expanding. Generally but not necessarily, self-expanding ring-shaped support members are preferred, as deployment of such does not require the use of often large and unmaneuverable expanding devices such as balloon catheters.

Deployment of a graft-stent assembly of the present invention is preferably performed according to the methods known in the art, for example as described in U.S. Pat. No. 5,723,004 or PCT Patent application PCT/IB98/00496 published as WO 99/15103 of the inventor. Deployment of a stent assembly 84 into a vessel 10 as depicted in FIG. 6A is discussed in detail hereinbelow, where first stent 64 and second stent 66 are non-self expanding.

In brief, according to one method of deploying a graft-stent assembly of the present invention, two guidewires are navigated through the body, up through a trunk vessel to be treated, and past bifurcation point 16, a first guidewire in trunk vessel 12 and a second guidewire into branch vessel 14. Thereafter, graft-stent assembly 84 with both first stent 64 and second stent 66 in the unexpanded state is mounted on a delivery system including one delivery catheter for the trunk part of graft-stent assembly 84 and a delivery catheter for the branch part of graft stent assembly 84, preferably including one balloon catheter for the trunk part and a transfer catheter for the branch part. If tubular graft wall 32 is provided with an expandable ring-shaped support member 44, it is preferred that a balloon catheter is also provided on the delivery catheter for the branch part.

The delivery catheter on which the trunk part of graft-stent assembly 84 is mounted is positioned over the first guidewire and the delivery catheter on which the branch part of graft-stent assembly 84 is mounted is positioned over the second guidewire. The two delivery catheters are advanced along the respective guidewires and thus navigated through the body to the proper location in vessel 10, first stent 64 between line A-A and bifurcation point 16, second expandable stent 66 between bifurcation pont 16 and line B-B and tubular graft wall 32 into branch vessel 14.

When proximal graft end 34 of tubular graft wall 32 is positioned properly across bifurcation 16, first expandable stent 24 and second expandable stent 26 are expanded, whether simultaneously or serially (vide infra), each to an expanded state of a desired size. Expansion of a stent generally occurs by positioning an inflation device (such as the balloon of a balloon catheter) inside the bore of a respective stent, inflating the balloon to a desired extent so as to expand the stent to a desired extent, and susequently deflating the balloon for withdrawal from the body.

In an embodiment where a delivery device includes a single balloon catheter for both stents, the balloon is generally already properly located within the bore of both stents. First expandable stent 24 and second expandable stent 26 are simultaneously expanded to a desired extent by inflation of the balloon and the balloon deflated.

In an embodiment where a delivery device includes a balloon catheter for each stent, the balloons are generally already properly located within the bore of a respective stent. First expandable stent 24 and second expandable stent 26 are either simultaneously or, preferably, serially expanded to a desired extent by inflation of the respective balloons and the balloons deflated.

In embodiments of the present invention where tubular graft wall 32 is provided with a self-expanding ring-shaped support member 44, then when ring-shaped support member 44 is properly positioned in branch vessel 14 ring-shaped support member 44 is allowed or induced to expand.

In embodiments of the present invention where tubular graft wall 32 is provided with a ring-shaped support member 44 that is not self-expanding, then when ring-shaped support member 44 is properly positioned in branch vessel 14, ring-shaped support member 44 is expanded using an expansion device such as a balloon of a balloon catheter. In some embodiments, the branch part of the delivery catheter is provided with a balloon on which the ring-shaped support member is mounted. When ring-shaped support member 44 is properly positioned in branch vessel 14, the ballon is activated to expand ring-shaped support member 44. In some embodiments, ring-shaped support member 44 is positioned using a delivery catheter not provided with an expansion device. After positioning of ring-shaped support member 44, the delivery catheter is withdrawn and an expansion device, such as a balloon catheter is advanced along the guidewire and threaded into the bore of ring-shaped support member 44. The expansion device is activated to expand ring-shaped support member 44.

In embodiments of the present invention where tubular graft wall 32 is provided with an expandable ring-shaped support member 44, ring-shaped support member 44 is positioned in branch vessel 14, and subsequently allowed or induced to expand.

In some embodiments where tubular graft wall 32 is not provided with an expandable ring-shaped support member 44 (e.g., FIGS. 4A-4F and FIGS. 5A-5F), then subsequent to stent expansion, the expansion balloons and catheters are withdrawn. The blood that then flows through branch vessel 14 stretches and presses tubular graft wall 32 against the walls of branch vessel 14 to conform thereto.

In some embodiments where tubular graft wall 32 is not provided with an expandable ring-shaped support member 44 (e.g., FIGS. 4A-4F and FIGS. 5A-5F), a ring-shaped support member 44 that is not attached to tubular graft wall 32 is used to hold distal end 36 of tubular graft wall 32 in place. In such embodiments, a ring-shaped support member 44 is place on the delivery catheter together with tubular graft wall (preferably inside tubular graft wall 32, preferably proximately to distal end 36, preferably on a delivery catheter provided with an expansion balloon) and tubular graft wall 32 and ring-shaped support member 44 are copositioned and codeployed. In embodiments, subsequent to deployment of tubular graft wall 32 in a branch vessel 14 and removal of the delivery catheter, a ring-shaped support member 44 is mounted on a delivery catheter in an unexpanded state and using an in place guide-wire, advanced into position, generally proximately to distal end 36 of tubular graft wall 32. When in position, ring shaped support member 44 is expanded or induced to expand, so as to hold distal end 36 of tubular graft wall 32 in place.

In embodiments where a delivery device already includes one or two balloon catheters on which the expandable stent or stents are mounted and a transfer catheter (without a balloon) on which non-self expanding ring-shaped support member is mounted, a balloon is generally already properly located within the bore of one of the expandable stents. In a first step, the balloon is inflated to expand the stent or stents of the trunk part of the graft-stent assembly to a desired extent and deflated. In a second step, the transfer catheter is withdrawn and a replacement balloon catheter advanced over the second guidewire until the balloon thereon is properly positioned inside ring shaped support member 44. The balloon of the replacement balloon catheter is inflated to expand ring shaped support member 44 to a desired extent and deflated. The two catheters are withdrawn from the body.

Deployment of other graft-stent assemblies of the present invention and in vessels such as 10 depicted in FIG. 1A or 18 depicted in FIG. 1B, or when one or more stents are self-expanding is understood by one skilled in the art upon perusal of the description herein.

Although described with respect to treating bifurcated vessels of the cardiovascular system, and especially bifurcated arteries, the teachings of the present invention are generally applicable to many different cardiovascular and non-cardiovascular applications. Specific cardiovascular applications include but are not limited to the deployment of a bifurcated stent of the present invention in ectatic arteries and ectatic arteries containing an obstructive lesion, aneurismatic arteries, saphenous vein grafts and native arteries, coronary perforation, coronary fistula, ostial coronary lesions, aortic abdominal aneurysm and other aneurismatic peripheral arteries, transjugular intrahepatic portal shunt, percutaneous transluminal angioplasty, fistula closing and neuro interventions (such as aneurysms and arterial-venous malformations), small vessel intraluminal grafting, and ostial renal artery lesions. Additional non-cardiovascular applications include but are not limited to urological, gastroenterological, respiratory and neurological applications.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A graft-stent assembly, comprising:
a) a flexible graft including a substantially tubular graft wall, a proximal graft end, a distal graft end and a graft bore; and
b) a substantially tubular first expandable stent having a first end, a second end and a bore in fluid communication with said graft bore through said proximal graft end;
wherein said graft bore and said bore of said first stent are discontinuous.

2. The graft-stent assembly of claim 1, wherein said graft bore substantially defines a branch part of the graft-stent assembly and said bore of said first stent defines in part a trunk part of the graft-stent assembly.

3. The graft-stent assembly of claim 1, wherein said graft bore diverges from said bore of said first stent at an angle of greater than about 5°.

4. The graft-stent assembly of claim 1, further comprising:
c) an expandable ring-shaped support member functionally associated with said graft through said distal graft end, configured to radially expand from an unexpanded state.

5. The graft-stent assembly of claim 4, wherein said graft bore and said ring-shaped support member substantially define a branch implant of the graft-stent assembly and said bore of said first stent substantially defines a trunk implant of the graft-stent assembly.

6. The graft-stent assembly of claim 4, wherein part of said graft wall is threaded through said ring-shaped support member.

7. The graft-stent assembly of claim 6, wherein said distal graft end is threaded through said ring-shaped support member and folded thereover.

8. The graft-stent assembly of claim 4, wherein said ring-shaped support member is substantially entirely contained within said graft bore.

9. The graft-stent assembly of claim 4, wherein said ring-shaped support member is located inside the bore of said graft wall and distal graft end is folded thereover.

10. The graft-stent assembly of claim 4, wherein said proximal graft end does not extend beyond said ring-shaped support member.

11. The graft-stent assembly of claim 4, wherein said ring-shaped support member does not significantly extend beyond said proximal graft end.

12. The graft-stent assembly of claim 4, wherein the axial length of said ring-shaped support member is substantially shorter than the axial length of said graft wall.

13. The graft-stent assembly of claim 12, wherein said axial length of said ring-shaped support member is less than about 30% of the axial length of said graft wall.

14. The graft-stent assembly of claim 4, wherein said ring-shaped support member is secured to said distal graft end.

15. The graft-stent assembly of claim 14, wherein said ring-shaped support member is secured to said distal graft end at a plurality of locations.

16. The graft-stent assembly of claim 14, wherein said ring-shaped support member is configured to clamp over said distal graft end at a plurality of locations on said distal graft end.

17. The graft-stent assembly of claim 16, wherein a part of said ring-shaped support member is bent about said distal graft end so as to clamp said distal graft end.

18. The graft-stent assembly of claim 1, wherein said graft-bore diverges from a side of said first stent.

19. The graft-stent assembly of claim 18, wherein said graft bore substantially defines a branch implant of the graft-stent assembly and said bore of said first stent substantially defines a trunk implant of the graft-stent assembly.

20. The graft-stent assembly of claim 18, wherein said first stent is provided with a side opening through which said graft bore is in fluid communication with said bore of said first stent.

21. The graft-stent assembly of claim 18, further comprising at least one connecting tab connecting said graft to said first stent.

22. The graft-stent assembly of claim 21, comprising at least two said connecting tabs.

23. The graft-stent assembly of claim 18, further comprising a second substantially tubular graft part having a bore not parallel to said graft bore connecting said graft to said first stent.

24. The graft-stent assembly of claim 23, wherein said second substantially tubular graft part surrounds at least part of said first stent.

25. The graft-stent assembly of claim 23, wherein said first stent is substantially entirely contained within said second substantially tubular graft part.

26. The graft-stent assembly of claim 23, wherein at least one end of said first stent emerges from said second substantially tubular graft part.

27. The graft-stent assembly of claim 23, wherein two ends of said first stent emerge from said second substantially tubular graft part.

28. The graft-stent assembly of claim 23, wherein at least part of said second substantially tubular graft part is disposed within said bore of said first stent.

29. The graft-stent assembly of claim 23, wherein said second substantially tubular graft part is substantially entirely disposed within said bore of said first stent.

30. The graft-stent assembly of claim 23, wherein a first end of said second substantially tubular graft part emerges from said first end of said first stent.

31. The graft-stent assembly of claim 30, wherein a first end of said second substantially tubular graft part emerges from said first end of said first stent and is folded thereover.

32. The graft-stent assembly of claim 30, wherein a second end of said second substantially tubular graft part emerges from said second end of said first stent.

33. The graft-stent assembly of claim 32, wherein a second end of said second substantially tubular graft part emerges from said second end of said first stent and is folded thereover.

34. The graft-stent assembly of claim 1, wherein said graft includes a second substantially tubular graft part having a first end, a second end and a bore not parallel to said graft bore connecting said graft to said first stent through said first end of said second substantially tubular graft part, and further comprising:
d) a second expandable stent having a first end, a second end and a bore in fluid communication with said graft bore connected to said graft through said second end of said second substantially tubular graft part.

35. The graft-stent assembly of claim 34, wherein said graft bore substantially defines a branch implant of the graft-stent assembly and wherein said bore of said first stent, said bore of said second stent and said bore of said second substantially tubular graft part substantially define a trunk implant of the graft-stent assembly.

36. The graft-stent assembly of claim 34, wherein said second substantially tubular graft part surrounds at least part of said first stent.

37. The graft-stent assembly of claim 34, wherein said first stent is substantially entirely contained within said second substantially tubular graft part.

38. The graft-stent assembly of claim 34, wherein said second end of said first stent emerges from said second substantially tubular graft part.

39. The graft-stent assembly of claim 34, wherein said second substantially tubular graft part surrounds at least part of said second stent.

40. The graft-stent assembly of claim 34, wherein said second stent is substantially entirely contained within said second substantially tubular graft part.

41. The graft-stent assembly of claim 34, wherein said second end of said second stent emerges from said second substantially tubular graft part.

42. The graft-stent assembly of claim 34, wherein at least part of said second substantially tubular graft part is disposed within said bore of said first stent.

43. The graft-stent assembly of claim 34, wherein a first end of said second substantially tubular graft part emerges from said second end of said first stent.

44. The graft-stent assembly of claim 34, wherein a first end of said second substantially tubular graft part emerges from said second end of said first stent and is folded thereover.

45. The graft-stent assembly of claim 34, wherein at least part of said second substantially tubular graft part is disposed within said bore of said second stent.

46. The graft-stent assembly of claim 34, wherein a first end of said second substantially tubular graft part emerges from said second end of said second stent.

47. The graft-stent assembly of claim 34, wherein a first end of said second substantially tubular graft part emerges from said second end of said second stent and is folded thereover.

48. The graft-stent assembly of claim 34, further comprising:
c) an expandable ring-shaped support member functionally associated with said graft through said distal graft end, configured to radially expand from an unexpanded state.

49. The graft-stent assembly of claim 48, wherein said graft bore and said ring-shaped support member substantially define a branch implant of the graft-stent assembly and wherein said bore of said first stent, said bore of said second stent and said bore of said second substantially tubular graft part substantially define a trunk implant of the graft-stent assembly.
